(19) United States
(12) Patent Application Publication
(10) Pub. No.: US 2025/0387059 A1
(43) Pub. Date: Dec. 25, 2025

(54) CONDUCTIVE TRANSFER

(71) Applicant: CONDUCTIVE TRANSFERS LIMITED, Barnsley (GB)

(72) Inventors: Vimanyu BEEDASY, Barnsley (GB); Paul Timothy BROOK, Barnsley (GB); Mark John CATCHPOLE, Saxmundham (GB); Steven Paul SUTCLIFFE, Rotherham (GB)

(73) Assignee: CONDUCTIVE TRANSFERS LIMITED, Barnsley (GB)

(21) Appl. No.: 18/877,468

(22) PCT Filed: Jun. 20, 2023
(86) PCT No.: PCT/GB2023/000035

§ 371 Date: Dec. 20, 2024

(30) Foreign Application Priority Data
Jun. 20, 2022 (GB) 2209030.2

(51) Int. Cl. A61B 5/256; A61B 5/00; A61B 5/28; H01B 7/02; H01B 7/40; H01B 13/06

(52) U.S. Cl. CPC A61B 5/256; A61B 5/0006; A61B 5/28; H01B 7/0225; H01B 7/40; H01B 13/06

(57) ABSTRACT

A conductive transfer (400, FIG. 4) comprises a conductive layer (404, FIG. 4) positioned between a first encapsulating layer (402, FIG. 4) and a second encapsulating layer (403, FIG. 4) forming at least one electrode (501) having a contact surface (503). The conductive transfer further comprises an adhesive layer (405, FIG. 4) for attaching the conductive transfer to a wearable item (102, FIG. 1). The at least one electrode comprises at least one raised protrusion (502) on the contact surface.

### CROSS REFERENCE TO RELATED APPLICATIONS

This application claims priority from United Kingdom Patent Application number GB 22 09 030.2, filed on 20 Jun. 2022, the whole contents of which are incorporated herein by reference.

### TECHNICAL FIELD

The present invention relates to a conductive transfer, a wearable item and a method of manufacturing a wearable item.

### BACKGROUND OF THE INVENTION

Medical conditions are often monitored by devices which incorporate sensors and similar to record data such as heart rates or similar. In many devices, a plurality of sensors is included and applied to a patient's skin to record the data. One such example is an ECG (electrocardiogram) monitor which utilises sensors to produce an ECG to monitor the heart.

In order to affix the sensors to a patient, the sensors used are typically wet electrodes which comprise a conductor and a gel material, for example hydrogel, which is used to contact the skin. Such electrodes and the corresponding gel cause skin irritation and often require a smooth contact on the patient's skin, meaning body hair needs to be removed to ensure adequate contact.

The positioning of these electrodes is critical for obtaining high quality data, such as a high-quality ECG, so the positioning is typically done by a highly trained clinician. Unfortunately, the electrodes must be removed for patient washing which limits the monitoring period to a day or two after which the electrodes must be replaced and positioned by such a professional. This is problematic when looking to capture occasional arrythmias, for example.

A further issue with such devices is that they often comprise a large number of electronic wires in combination with an electronic box which is carried around the neck. When patients are required to wear such devices over an extended period, the devices can be restrictive and uncomfortable.

There remains a need to provide a wearable item which is comfortable for a user to wear and which enables recording of medical data and overcomes the aforesaid issues.

### BRIEF SUMMARY OF THE INVENTION

According to a first aspect of the present invention, there is provided a conductive transfer, comprising: a conductive layer positioned between a first encapsulating layer and a second encapsulating layer forming at least one electrode having a contact surface; an adhesive layer for attaching said conductive transfer to a wearable item; wherein said at least one electrode comprises at least one raised protrusion on said contact surface.

According to a second aspect of the present invention, there is provided a method of manufacturing a wearable item comprising a conductive transfer, comprising the steps of: printing a first encapsulating layer and a second encapsulating layer and a conductive layer between said first encapsulating layer and said second encapsulating layer to form at least one electrode having a contact surface; providing a wearable item; locating said at least one electrode in position on said wearable item; attaching said at least one electrode to said wearable item; printing an embossing layer onto said contact surface of said electrode; and embossing said at least one electrode to produce at least one raised protrusion in said embossing layer on said contact surface.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows a wearable item comprising a conductive transfer worn by a user;

FIG. 2 shows a schematic layout of a conductive transfer in accordance with the invention;

FIG. 3 shows an example electrode of the conductive transfer of FIG. 2;

FIG. 4 shows an embodiment of an ink stack of the conductive transfer of FIGS. 2 and 3;

FIG. 5 shows an example embodiment of a conductive transfer having raised protrusions in accordance with the invention;

FIG. 6 shows an example mould for production of the conductive transfer of FIG. 5;

FIGS. 7A and 7B show a plan view and a cross-sectional view of example moulds for production of conductive transfers with raised protrusions;

FIG. 8 shows a schematic of a manufacturing process for creating raised protrusions on a conductive transfer;

FIG. 9 shows a method of manufacturing a wearable item comprising a conductive transfer; and

FIG. 10 shows a schematic of a wearable item and conductive transfer when worn by a user.

### DETAILED DESCRIPTION OF EMBODIMENTS OF THE INVENTION

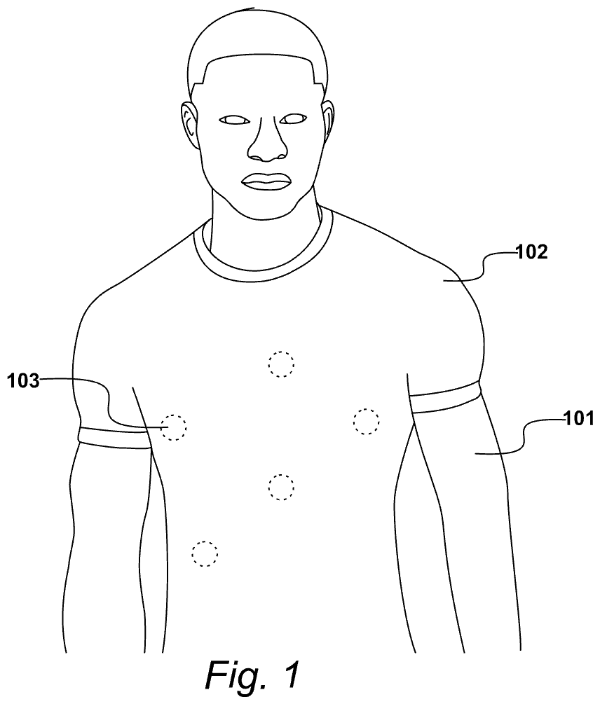

### FIG. 1

A wearable item in accordance with the present invention is shown in FIG. 1.

A user or wearer 101 is shown wearing wearable item 102. In the embodiment, wearable item 102 comprises a conductive transfer 103 incorporated into the fabric of wearable item 102.

In the embodiment, wearable item 102 comprises a compression fabric which is configured to be worn close to the wearer's 101 body. These types of compression fabrics are known for being stretchable and close-fitting and are often used in sportswear applications. Compression fabrics of this type typically comprise spandex fibres or elastane fibres and another material such as nylon or polyester. One example of such a compression fabric is sold under the tradename Lycra®.

In the embodiment, user 101 wears wearable item 102 in an application for measuring vital signs to produce an electrocardiogram. In this way, wearer 101 wears wearable item 102 such that conductive transfer 103 in wearable item 101 is configured to produce appropriate data to result in an electrocardiogram.

In a further embodiment, a substantially similar conductive transfer may be incorporated into a wearable item to provide alternative measurements to that of an electrocardiogram. In one such example, a conductive transfer is configured to provide electrical muscle stimulation (EMS) to a wearer of the wearable item. It is appreciated that, in further embodiments, the conductive transfer described herein may be suitably incorporated into wearable items for alternative medical or health applications or other purposes. This includes but is not limited to uses in relation to transcutaneous electrical nerve stimulation (TENS), electrical impedance tomography (EIT), electrotherapy for wound healing, electromyography (EMG), electroencephalography (EET), electrogastrography (EGG) and electroglottography (EGG).

Conductive transfer 103 is shown schematically and represented by a plurality of electrodes included in wearable item 102. In the embodiment where the conductive transfer is configured to measure vital signs to produce an electrocardiogram, it is preferable to arrange a plurality of electrodes in a manner consistent with the wearer's body shape and parts of the body in which measurements should be made. This is known as the EASI electrode placement where E represents a position at the level of the fifth of the lower sternum, A represents an electrode positioned in the left midaxillary line at the same level as E, S represents an electrode positioned on the manubrium sterni and I represents an electrode positioned in the right midaxillary lines at the same level as E. A further ground electrode can be placed at any convenient location for use in conductive transfer 103.

In the embodiment, wearable item 102 is shown as a T-shirt. It is appreciated that however, that any other suitable wearable item may be utilised in alternative embodiments. This includes, but is not limited to, shirts, long-sleeved T-shirts, shorts, dresses, underwear including bras and sports bras or other wearable items or armbands, wristbands or similar which may be appropriate for measuring certain medical data.

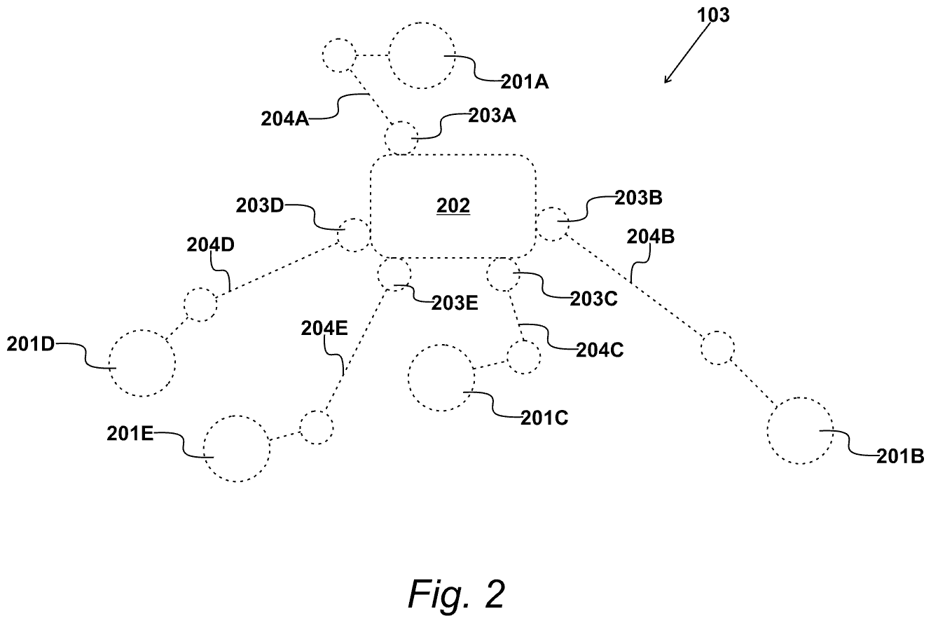

### FIG. 2

A schematic layout of conductive transfer 103 is shown in isolation in FIG. 2.

Conductive transfer 103 comprises at least one electrode and, in this embodiment, comprises a plurality of electrodes 201. In this illustrated embodiment, electrodes 201A, 201B, 201C and 201D are arranged in the EASI electrode placement configuration consistent with measurements for an electrocardiogram as described previously. The fifth electrode 201E of this five-electrode arrangement is arranged as a ground electrode.

The arrangement of conductive transfer 103 further comprises an integrated circuit (IC) chip 202 comprising a processor, analogue-to-digital converter (ADC) and a wireless connection. In an embodiment, integrated circuit 202 comprises a plurality of chips and, in this embodiment, the processor, analogue-to-digital converter (ADC) and wireless connection are provided as separate chips. In addition, further discrete components such as crystals and passive components may be provided on separate chips.

In an embodiment, the integrated circuit may comprise a flexible printed circuit board (PCB) or a rigid printed circuit board (PCB). Thus, this arrangement may further be configured to form a hybrid electronic circuit and provide additional connections to further conductive transfers with alternative or similar functionalities.

In the embodiment, the digital data provided from the ADC may be viewed as live ECG data on an electronic device having a wireless receiver. Example electronic devices include a personal computer or mobile electronic device such as a tablet computer. In this way, a medical professional may monitor the data in real-time.

In a further embodiment, the digital data may be processed remotely and transmitted to cloud storage for processing, analysis and storage. It is anticipated that this storage may comprise part of a patient record system or similar.

In an alternative embodiment, the processor of integrated circuit chip 202 is configured to process digital data within chip 202 and consequently conductive transfer 103. In an embodiment, this process comprises the use of machine learning and artificial intelligence and an artificial neural network configured to identify patterns in output data. This advantageously may reduce the amount of data for transmission wirelessly thereby reducing the power consumption in respect of conductive transfer 103.

In the embodiment, the wireless connection may comprise a Wi-Fi module, a Bluetooth module or any other suitable wireless connection. The wireless connection enables the data obtained by means of conductive transfer 103 to be transmitted to an electronic device for analysis by a medical professional. In an embodiment, processing of data is conducted within the chip 202 to enable processing to occur on a wearable item, such as wearable item 202. In an alternative embodiment, the wireless connection enables data to be transmitted remotely such that processing from the conductive transfer can be conducted remotely in a storage cloud.

In the embodiment, the IC chip 202 comprises electrode connectors 203 which correspond to each electrode 201. Thus, the processor is therefore electrically connected to each electrode. In addition, each electrode 201 is electrically connected to processor 202 by means of a conductive track 204 which connects each electrode 201 to its corresponding connector electrode 203 and consequently the IC chip, processor and ADC thereby enabling signals received from each electrode 201 to be processed appropriately and wirelessly transmitted for medical assessment or analysis.

In the embodiment, conductive transfer 103 comprises a plurality of conductive tracks 204 of which each comprises a printed conductive ink. In an embodiment, the printed conductive ink comprises a silver-based ink. It is appreciated however that other conductive inks may be utilised if these are deemed appropriate in specific embodiments.

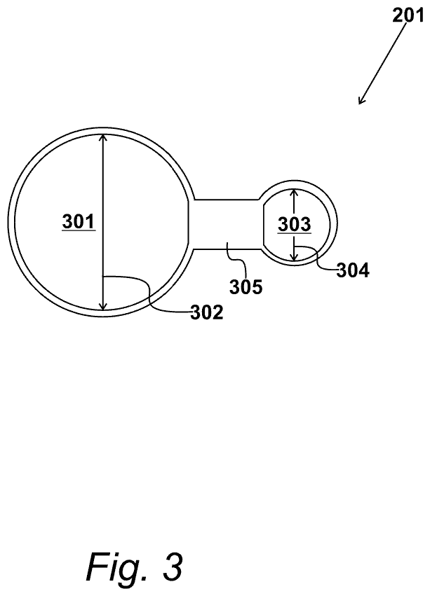

### FIG. 3

An example electrode is shown in FIG. 3 in plan view which may be utilised in the manner previously described.

In the embodiment, electrode 201 comprises a first circular portion 301 which presents a first diameter 302. A second circular portion 303 comprises a second diameter 304. Portion 301 and portion 302 are connected by via a track 305. Portion 301 and portion 303 each comprise a conductive ink which may be a silver-based ink or any other suitable conductive material. In the embodiment, diameter 302 is larger than diameter 304. In a specific embodiment, diameter 302 is thirty millimetres (30 mm) and diameter 304 is ten millimetres (10 mm).

The conductive ink of portions 301 and 303 are provided with an opening in the electrode such that the conductive material is exposed to enable an electrical contact to be provided when in use. Track 305 alternatively comprises conductive ink encapsulated within an encapsulating material in order to increase the durability and protection of the track 305. In the embodiment, track 305 typically measures ten millimetres (10 mm) in length and four millimetres (4 mm) in width. It is appreciated that the dimensions indicated herein are examples and the invention is not limited to a specific size of electrode and can be scaled accordingly dependent on the application.

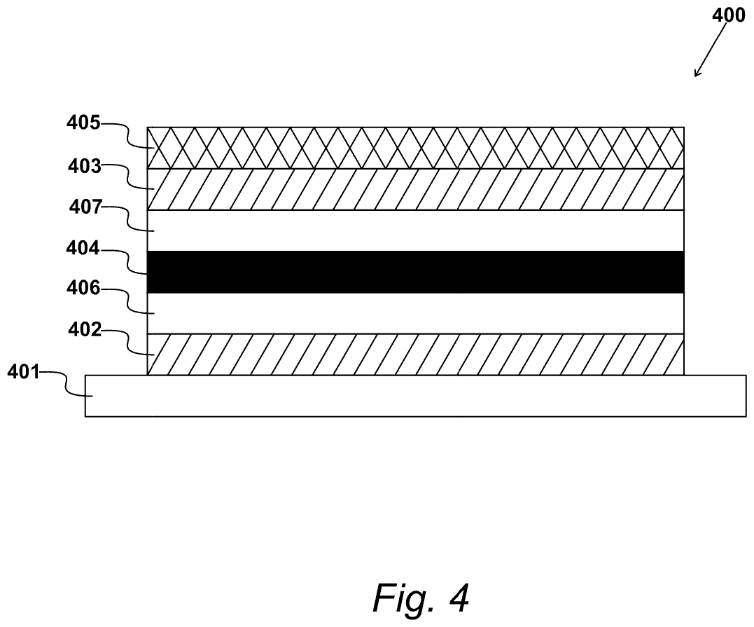

### FIG. 4

In the embodiment, electrodes 201 comprise a plurality of layers which are formed from printed inks to enable the conductive transfer to be applied to a suitable wearable item such as wearable item 102. In FIG. 4, an example ink stack illustrating the layers of the conductive transfer (and consequently the electrodes) is shown in schematic cross-sectional view. It is appreciated that in practice the thickness of the layers in total is in the region of microns such that, when fitted to a wearable item, conductive transfer 103 is not noticeable by a user when wearing the wearable item beyond the normal awareness of a typical wearable garment.

In the embodiment of FIG. 4, conductive transfer 400 comprises a transfer film 401 onto which a first encapsulating layer 402 is printed. In the embodiment, conductive transfer 400 further comprises a second encapsulating layer 403 and a conductive layer 404 which is positioned between the first encapsulating layer 402 and the second encapsulating layer 403. Together, these layers are configured to form at least one electrode in the conductive transfer 400. In the embodiment, to enable conductive transfer 400 to be attached to a wearable item, conductive transfer 400 further comprises an adhesive layer 405 which is suitable for attaching the conductive transfer to a wearable item such as wearable item 102.

In the embodiment, conductive transfer 400 further comprises a first reinforcing layer 406 and a second reinforcing layer 407. In the embodiment, reinforcing layer 406 is positioned between encapsulating layer 402 and conductive layer 404. Reinforcing layer 407 is similarly positioned between conductive layer 404 and encapsulating layer 403.

In manufacture, each of the layers may be printed sequentially onto transfer film 401 by printing encapsulating layer 402 followed by reinforcing layer 406, followed by conductive layer 404, reinforcing layer 407, encapsulating layer 403 and adhesive layer 405.

In the embodiment, any one of the layers of the conductive transfer may be printing utilising any suitable printing method. For example, suitable printing methods include screen-printing, inkjet printing, 3D printing, gravure printing and offset printing. In addition, any of the printing methods may be produced in sheet-to-sheet formats or roll-to-roll formats as required.

In the embodiment, and, with reference to FIG. 3, encapsulating layers 402 and 403, adhesive layer 405 and reinforcing layers 406 and 407 each comprise an opening such that conductive layer 404 is exposed prior to pressing for test purposes and after pressing to enable electrical connections to be provided. This ensures that any measurements made electrically can be recorded.

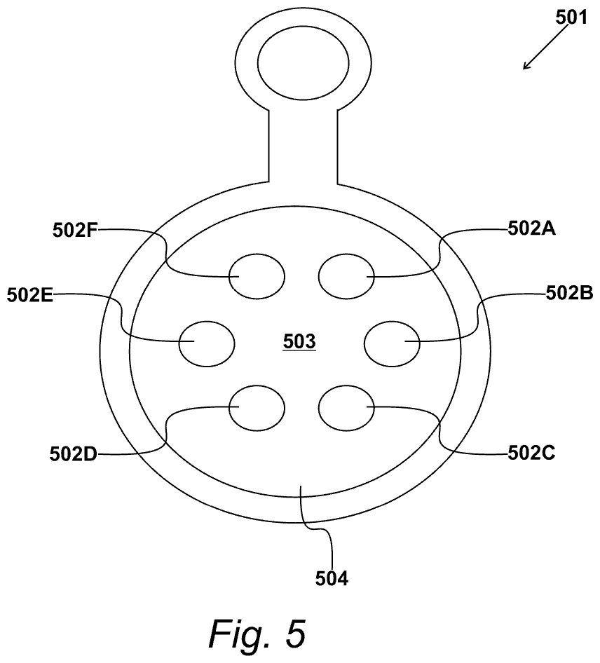

### FIG. 5

In accordance with the invention, the conductive transfer described previously comprises at least one electrode which comprises at least one raised protrusion on a contact surface. In an example embodiment shown in FIG. 5, an electrode 501 is shown which comprises a plurality of raised protrusions 502 on a contact surface 503.

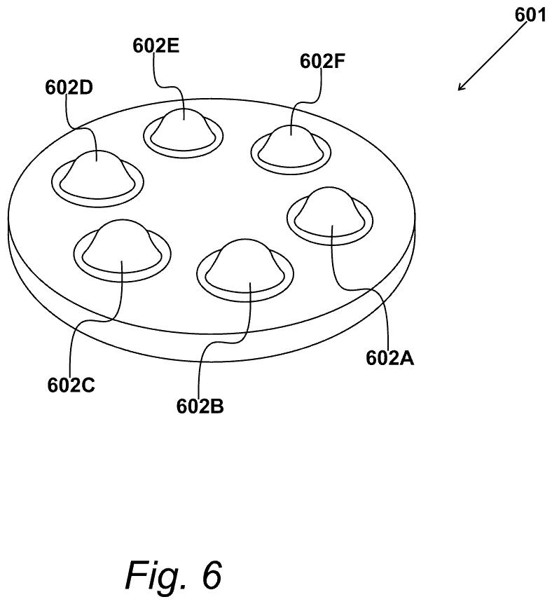

Raised protrusions 502 may be formed by an embossing process as will be described further with respect to FIGS. 6 to 9.

In the embodiment, electrode 501 is substantially similar to electrode 201 previously described in FIG. 3. In order to create the raised protrusions 502 on electrode 501 in addition to the encapsulating, reinforcing, adhesive and conductive layers previously described, an additional layer is included in order to enable the raised protrusions to be embossed into electrode 501. In the embodiment, this additional layer comprises a silicone layer formed from a printed silicone ink. It is appreciated however, that the layer comprising the raised protrusions may comprise an alternative suitable material to silicone provided the material provides suitable adhesion properties against a wearer's skin.

In the embodiment, raised protrusions 502 are included in the larger diameter first portion 504 to enable a contact to be made with a wearer's skin when wearing a suitable wearable item similar to wearable item 102.

Raised protrusions 502 consequently increase the surface area in relation to the contact surface thereby enabling an improved grip of the electrode 501 with a wearer's skin without the need for any gels or use of wet electrodes. Thus, in this way, a dry electrode is provided with raised protrusions which further assists in avoiding the issues experienced in conventional devices in relation to a wearer's body hair which can restrict the attachment of the electrodes.

### FIG. 6

The raised protrusions shown previously in relation to FIG. 5 are produced by means of an embossing process which will now be described further with respect to FIGS. 6 to 9.

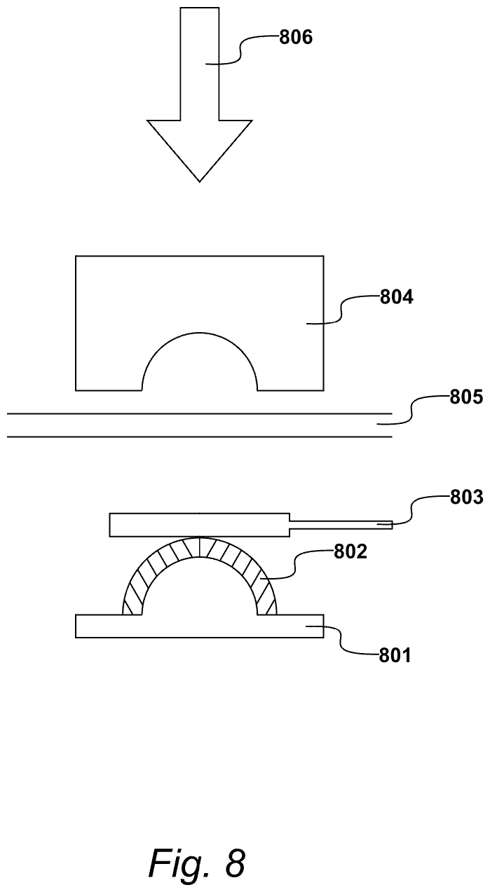

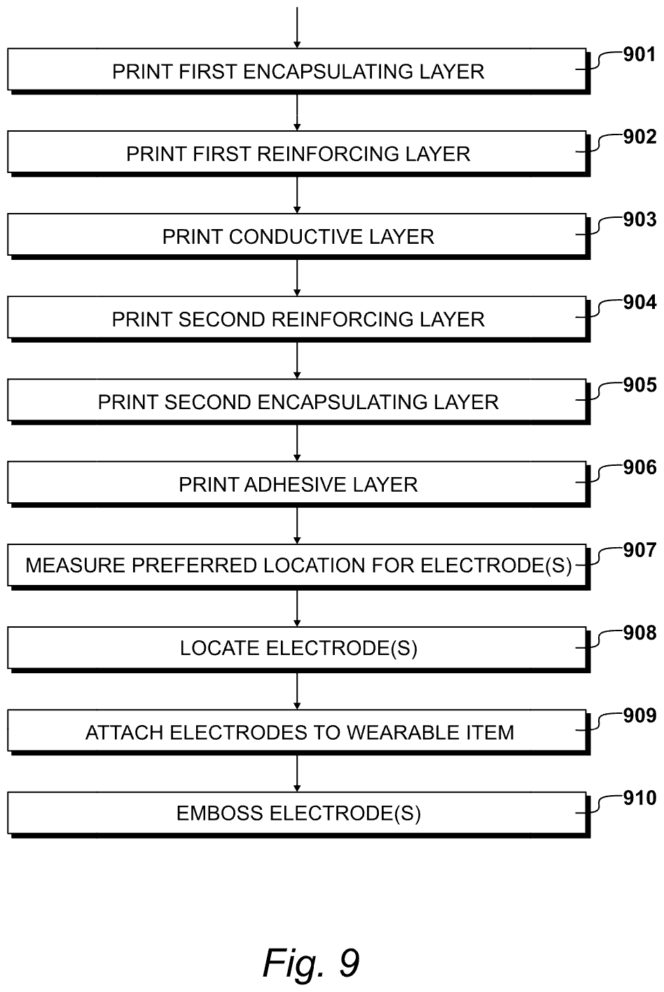

FIG. 6 shows an example male part of a mould for creating the embossed raised protrusions shown previously in FIG. 5. Mould 601 comprises a plurality of micropillars 602 which correspond with raised protrusions 502 shown previously. In order to create raised protrusions, which will be described in further detail in FIGS. 8 and 9, the conductive transfer previously described is positioned in a hot press between the male portion of mould 601 of FIG. 6 and a corresponding female portion. In this way, the conductive transfer is sandwiched between the two parts of mould 601 to ensure that the micropillars 603 deform conductive transfer to result in the corresponding plurality of raised protrusions.

In the embodiment, the micropillars are positioned circumferentially around mould 601 and, in an embodiment, include six micropillars. It is appreciated that, in alternative embodiments, an alternative plurality of micropillars resulting in an alternative plurality of raised protrusions may be utilised within a similar mould to create alternative raised protrusions depending on the requirements of the application in which the conductive transfer described herein is applied.

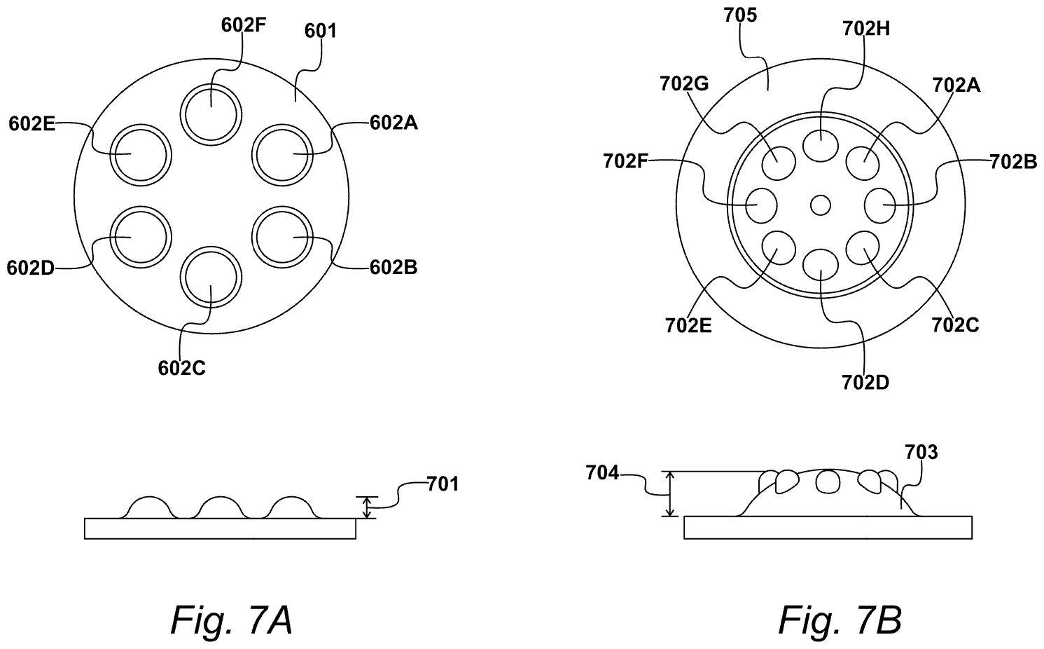

### FIG. 7A and FIG. 7B

FIG. 7 a illustrates a cross-sectional view and a plan view of a first embodiment of a mould in accordance with the invention. Similar cross-sectional and plan views of an alternative embodiment of a mould suitable for the utilising to create the raised protrusions on the conductive transfers described herein is further shown with respect to FIG. 7B.

The mould depicted in FIG. 7A is substantially similar to that depicted previously in FIG. 6. As shown, mould 601 in plan view illustrates six micropillars 602 which are spaced circumferentially around the diameter of mould 601.

In an embodiment, the diameter of each micropillar 602 is three point eight millimetres (3.8 mm) thereby resulting in a corresponding raised protrusion consistent with this diameter.

Referring to the cross-sectional view of FIG. 7A, the height 701 of the micropillars 602 is, in an embodiment, two millimetres (2 mm). This represents the depth at which the embossed raised protrusions are made in the conductive transfer.

With respect to the alternative embodiment shown in FIG. 7B, in this embodiment, micropillars 702 are arranged in an alternative manner and comprise eight micropillars. In this embodiment, the micropillars are positioned on a raised portion 703 having a total height of four millimetres (4 mm). This leads to alternative raised protrusions which have been shown to provide an effective output with in terms of the electrical conductivity of the conductive transfer. Furthermore, the raised portion of an embossed electrode allows improved electrical contact with a human body, particularly in undulations across the body surface, for example, in the location of the sternum.

In practice, experimental data using moulds 601 and 705 have shown that the variance between an electrode which has not been subjected to the embossing process and without raised protrusions, and an electrode which has been embossed to create the raised protrusions varies in resistance of between as little as zero point six Ohms (0.6Ω) indicating that the electrical conductivity can be maintained following the embossing process. In particular, it has been found that the mould of FIG. 7B is effective in maintaining the difference in resistance and maintaining the overall electrical conductivity of the conductive transfer to a minimum due to its higher radius of curvature which enables the conductive layer to stretch more evenly without forming any sharp edges. Consequently, the raised portion 703 of the mould of FIG. 7B provides a suitable solution for the embossing process.

While the mould of FIG. 7A corresponding showed a higher variance in electrical conductivity, this was still as little as two point five ohms (2.5Ω) which is considered to be within an acceptable range of electrical conductivity variance.

### FIG. 8

A schematic of the embossing process in which the embossing process is utilised to produce the raised protrusions on the conductive transfer by means of a mould, such as the mould shown in FIGS. 6 and 7 is shown schematically in FIG. 8.

In the embodiment, a male portion 801 of a mould is applied with a release agent 802. A conductive transfer electrode 803 is positioned above the release agent 802 having had a silicone layer printed thereon in the area to be embossed. Electrode 803 is then aligned with the female portion 804 of the mould. A layer of fabric 805 is included between the mould parts 801 and 804 to prevent abrasion on the conductive transfer.

Once positioned, conductive transfer 803 can be embossed by means of an application of heat and pressure 806 which may be applied by positioning mould parts 801 and 804 in a conventional heat press thereby bringing mould parts 801 and 804 together and consequently embossing the conductive transfer 803 to produce a conductive transfer having raised protrusions such as that shown in FIG. 5.

### FIG. 9

A method of manufacturing a wearable item comprising a conductive transfer is shown schematically with respect to FIG. 9. At step 901 a first encapsulating layer is printed onto the release film shown previously in FIG. 4.

If required, a first reinforcing layer may be printed on top of the first encapsulating layer at step 902. The reinforcing layers described herein are particularly useful in relation to the embossing process described in FIG. 8, however, it is appreciated that both the first and second reinforcing layers may be optional and an embossing process may be utilised which does not affect the nature of the raised protrusions in the absence of the reinforcing layers. In these cases, it is considered that the conductive transfer may be produced without the inclusion of the reinforcing layers.

At step 903 a conductive layer is printed which typically comprises a conductive silver ink printed on top of the first reinforcing layer. At step 904 a second reinforcing layer is printed above the conductive layer such that the conductive layer is sandwiched between the two reinforcing layers and protected by means of the reinforcing layers.

At step 905, a second encapsulating layer is printed thereby protecting the conductive layer in use, such as when being washed as part of a wearable item, rather than during the manufacturing process.

Once the second encapsulating layer has been printed and dried appropriately, an adhesive layer is printed at step 906 to complete the layers of the conductive transfer. Thus, by this stage a conductive transfer has been produced which forms at least one electrode having a contact surface which is now ready to be attached to a wearable item. The contact surface is the surface which is configured to be worn in contact with a wearer's skin.

A wearable item is therefore provided and, at step 907 preferred locations for any electrodes required are measured and identified for a specific user. In practice, this may entail a medical professional taking measurements from a patient to determine the precise location of an electrode to ensure that the most accurate measurements are made depending on the wearer or patient's particular medical condition. Once the preferred location has been identified and measured, at step 908 these positions can be consequently located onto the wearable item and any electrodes required can be positioned and located thereon.

Thus, once any electrodes are allocated in position on the wearable item, the electrodes may then be appropriately attached to the wearable item at step 909.

Step 910 allows for the electrodes to be appropriately embossed to enable more accurate alignment and suitable positioning on a user when wearing the wearable item. As part of the embossing process, the contact surface side (which ultimately contacts a wearer's skin in use) of the electrodes has a further layer overprinted with a suitable ink corresponding to the pattern of the required protrusions. That is, the pattern ensures that conductive areas of the conductive layers remain exposed to provide electrical contact, while the protrusions are positioned for adhesion to a wearer's skin without hindering the conductive contact. In the embodiment, the additional layer for embossing and providing the protrusions comprises a silicone layer.

In the embodiment shown, the step of embossing the electrodes, as described in FIG. 8, is shown as occurring after the electrodes have been attached to the wearable item. In this way, the wearable item itself also experiences the embossing process.

In an alternative embodiment, the electrode may be appropriately embossed prior to being attached to the wearable item. This may be more appropriate in some circumstances where the conductive transfer and electrodes are manufactured in a separate location to the wearable item itself. In this case, the wearable items incorporating the electrodes may be suitably configured to be attached at a later time. This may occur during steps 907 and 908 where the electrodes are located and the preferred location of the electrodes on the wearable item are determined by the medical professional or other.

Consequently, it may also be possible for the medical professional or, in some cases the wearer or other individual, to locate, position and, in addition, attach the electrodes and conductive transfer to the wearable item at step 909.

In a particular embodiment, the wearable item may comprise a connectable material and, a corresponding connectable material is provided as part of the conductive transfer such that the conductive transfer can be attached the wearable item by means of the connectable material and corresponding connectable material. The connectable material may be provided in the form of a fastening such as a press stud or may comprise a form of adhesive material. In a further embodiment, the connectable material comprises a hook and loop fastening.

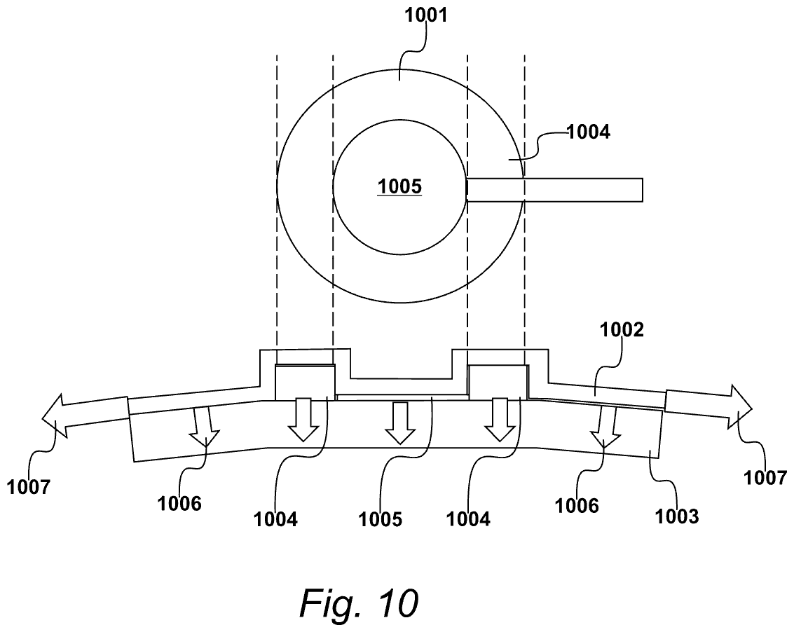

### FIG. 10

A schematic of a wearable item comprising a conductive transfer as described herein when worn by a user is shown in FIG. 10.

The schematic shows electrode 1001 in plan view aligned with a cross-sectional view of electrode 1001 in combination with the fabric of a wearable item 1002 and the skin 1003 of the wearer of wearable item 1003.

When worn by a user, the additional silicone layer of electrode 1001 provides raised protrusions 1004 in conductive transfer 1001. In use, conductive layer 1005 remains in contact with the skin 1003 on the contact surface to enable appropriate data to be recorded. In the schematic of FIG. 10 therefore, the conductive layer 1005 of conductive transfer 1001 is surrounded by raised protrusions 1004 on a contact surface thereby assisting in holding conductive transfer 1001 in place.

In use, the fabric of wearable item 1002 assists in providing compressive forces 1006 onto the wearer's skin 1003 while the stretchability of wearable item 1002 results in a force in tension across the wearer's skin 1003 which, in combination, ensure that conductive transfer 1001 is pulled towards the user to maintain a desirable contact. In addition, the raised protrusions 1004 ensure that the conductive transfer is pulled towards the user's skin to ensure any required readings can be made.

It is appreciated that, while the examples described herein relate to a medical device and reading of medical data, the conductive transfer described herein is not limited to this type of application, and may be utilised in, for example, leisure activities or other applications which require electrical outputs in wearable items.

1. A conductive transfer, comprising:
a conductive layer positioned between a first encapsulating layer and a second encapsulating layer forming at least one electrode having a contact surface, said second encapsulating layer including an opening that exposes said conductive layer to provide an electrical contact of said at least one electrode and said at least one electrode includes at least one raised protrusion on said contact surface and said at least one raised protrusion configured to provide grip for the electrode; and
an adhesive layer for attaching said conductive transfer to a wearable item.

2. The conductive transfer of claim 1, further comprising:
a first reinforcing layer positioned between said first encapsulating layer and said conductive layer; and
a second reinforcing layer positioned between said second encapsulating layer and said conductive layer.

3. (canceled)

4. The conductive transfer of claim 1, wherein said conductive transfer comprises a plurality of electrodes that includes said at least one electrode.

5. The conductive transfer of claim 1, wherein said conductive layer is a conductive ink layer, said first encapsulating layer is a first encapsulating ink layer, and said second encapsulating layer is a second encapsulating ink layer.

6. The conductive transfer of claim 1, further comprising a processor electrically connected to said electrode.

7. The conductive transfer of claim 6, further comprising an artificial neural network configured to identify patterns in output data.

8. The conductive transfer of claim 4, wherein each of said plurality of electrodes is connected by way of a conductive track.

9. The conductive transfer of claim 8, wherein said conductive track comprises a printed conductive ink.

10. A wearable item comprising:
a conductive transfer that comprises:
a conductive layer positioned between a first encapsulating layer and a second encapsulating layer forming at least one electrode having a contact surface said second encapsulating layer including an opening that exposes said conductive layer to provide an electrical contact of said at least one electrode and said at least one electrode includes at least one raised protrusion on said contact surface and said at least one raised protrusion configured to provide grip for the electrode; and
an adhesive layer for attaching said conductive transfer to the wearable item.

11. The wearable item of claim 10, said wearable item comprising a compression fabric.

12. The wearable item of claim 10, wherein said conductive transfer is configured to measure vital signs of a wearer of said wearable item to produce an electrocardiogram.

13. The wearable item of claim 10, wherein said conductive transfer is configured to provide any one of the following to a wearer of said wearable item:
electrical muscle stimulation; electrical nerve stimulation; electrical impedance tomography; electromyography; electroencephalography; electrotherapy in wound healing; electrogastrography; and electroglottography.

14. The wearable item of claim 10, wherein said conductive transfer further comprises a processor configured to process data when on said wearable item.

15. The wearable item of claim 10, wherein said conductive transfer further comprises a processor configured to transmit data from said conductive transfer for processing remotely in a storage cloud.

16. The wearable item of claim 10, further comprising a connectable material and said conductive transfer further comprises a corresponding connectable material, such that said conductive transfer is attached to said wearable item by said connectable material.

17. (canceled)

18. A method of manufacturing a wearable item comprising a conductive transfer, comprising:
printing a first encapsulating layer and a second encapsulating layer and a conductive layer between said first encapsulating layer and said second encapsulating layer to form at least one electrode having a contact surface, said second encapsulating layer printed to include an opening to expose the conductive layer and provide an electrical contact of said at least one electrode;
providing a wearable item;
locating said at least one electrode in position on said wearable item;
attaching said at least one electrode to said wearable item by an adhesive layer; and
embossing said at least one electrode to produce at least one raised protrusion configured to provide grip on said contact surface.

19. The method of claim 18, wherein said embossing is conducted following said attaching said at least one electrode to said wearable item.

20. The method of claim 18, wherein said embossing is conducted prior to said attaching said at least one electrode to said wearable item.

21. The method of claim 18, wherein the embossing produces said at least one raised protrusion by a mould.

22. The method of claim 18, wherein the of embossing comprises an application of heat and pressure to electrodes to produce said raised protrusion.

23. (canceled)
